(19) United States
(12) Patent Application Publication
(10) Pub. No.: US 2007/0191337 A1
(43) Pub. Date: Aug. 16, 2007

(54) Annellated carbamyl-aza-heterocyclic compounds, a focused library, pharmaceutical compositions, and methods of preparing the same

(76) Inventors: Alexander Ivashchenko, Encinitas, CA (US); Vladimir Vvedensky, Irkutsk (RU); Aleksei Ilyin, Moscow (RU); Volodymyr Kysil, Kiev (UA); Alexander Khvat, San Diego, CA (US); Yulia Kuzovkova, Moscow (RU); Sergey Kutepov, Ryazan (RU); Irina Dmitrieva, Moscow (RU); Denis Zolotarev, Moscow (RU); Sergey Tkachenko, Moscow region (RU); Llya Okun, San Diego, CA (US); Dmitri Kravchenko, Moscow region (RU); Vladimir Kobak, Moscow region (RU); Andrei Trifilenkov, Moscow (RU); Yulia Mishunina, Moscow region (RU); Marina Loseva, Moscow (RU); Elena Rizhova, Moscow (RU); Vladislav Parchinsky, Moscow (RU); Sergey Tsirulnikov, Moscow (RU); Alexandr Kyseley, San Diego, CA (US)

Correspondence Address: David B Waller;David B Waller & Associates, 5677 Oberlin Drive, Suite 214, San Diego, CA, 92121, US

(21) Appl. No.: 11/547,874

(22) PCT Filed: Apr. 29, 2005
(86) PCT No.: PCT/RU05/00235

§ 371 Date: Oct. 5, 2006

(30) Foreign Application Priority Data
Apr. 29, 2004 (RU) 20044113251

(51) Int. Cl. A61K 31/554; A61K 31/551; A61K 31/498; C07D 498/14; C07D 487/14

(52) U.S. Cl. 514/211.05; 514/219; 514/220; 514/249; 544/350; 540/490; 540/497

(57) ABSTRACT

This invention relates to novel annellated carbamyl-aza-heterocyclic compounds that are potential physiologically active compounds (agonists, antagonists, and receptor modulators, enzyme inhibitors, oncolytics, antibacterial and antiparasitic agents, and so on), to a focused library comprising annellated carbamyl-aza-heterocyclic compounds, a pharmaceutical composition comprising annellated carbamyl-aza-heterocyclic compounds as the active ingredient, and to methods of producing and using the same.
The invention relates to annellated carbamyl-aza-heterocyclic compounds of general formula 1: 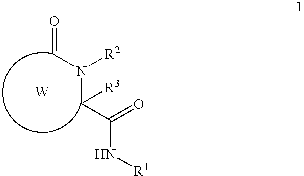 wherein: W is 6-oxopiperazine, [1,4]diazepan, [1,4]thiazepan or [1,4]oxazepan compound annellated to at least one optionally substituted and optionally condensed heterocyclic compound Q;
R¹, R² and R³ are, independently of one another, a hydrogen atom, an inert substituent, an optionally substituted C₁-C₆ alkyl, an optionally substituted C₃-C₈ cycloalkyl, an optionally substituted phenyl, an optionally substituted aryl, or an optionally substituted heterocyclyl; and Q is a pyrrole, pyrazole, imidazole, thiazole, pyrrolidine, indole, benzofuran, 4,5,6,7-tetrahydrobenzothiophene, thieno[3,2-b]pyrrole, furo[3,2-b]pyrrole, thieno[2,3-b]pyrrole, benzimidazole, pyridine, quinoline, or 1,2,3,4-tetrahydroisoquinoline cyclic compound.

### FIELD OF THE INVENTION

This invention relates to synthesis of new chemical compounds, a search for new physiologically active compounds, leader compounds, and drug candidates produced by screening combinatorial or focused libraries of compounds, and to a pharmaceutical composition, and to methods of producing and using the same.

More specifically, this invention relates to novel annellated carbamyl-aza-heterocyclic compounds that are interesting as potential physiologically active compounds (agonists, antagonists, and receptor modulators, enzyme inhibitors, oncolytics, antibacterial and antiparasitic agents, and so on), to a focused library comprising annellated carbamyl-aza-heterocyclic compounds, to a pharmaceutical composition comprising annellated carbamyl-aza-heterocyclic compounds as an active ingredient, and to method of preparing and using the same.

### BACKGROUND OF THE INVENTION

There is a large number of natural and synthetic physiologically active compounds having molecules that are annellated aza-heterocyclic compounds. Among natural compounds, the more significant are anti-neoplastic and antibacterial alkaloids such as longamide, longamide B, and phakellstatins derived from sea sponges Agelas genus, Homaxinella sp. and Phakellia mauritiana [Cafieri, F., et al., J. Nat. Prod. 1998, 61: 122. Umeyama, A., et al., J. Nat. Prod. 1998, 61: 1433. Poullennec, K., Romo, D. J. Am. Chem. Soc. 2003, 125: 6344]

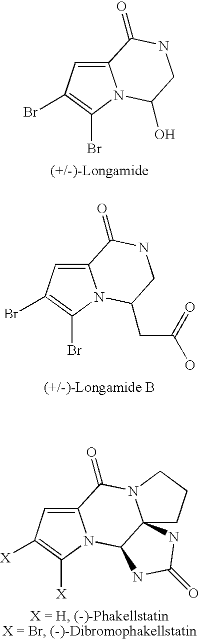

Synthetic annellated carbamyl-aza-heterocyclic compounds have an extraordinarily broad spectrum of physiological activity. For example, 4-methyl-2-(3-trifluoromethyl phenyl)-hexahydropyrido[1,2-a]pyrazin-3-one exhibits the properties of an anxiolytic and antidepressant [Silvestrini, B., Baiocchi, L. ACRAF SpA. WO 8705022, 1987], 3-[1-oxo-2-[2-(piperidin-1-yl)ethyl]-1,2,3,4-tetrahydro-pyrrolo[n1,2-α]pyrazin-7-yl-carboxamido]propionic acid is an antagonist of the fibrinogenic gpIIb/IIIa receptor and, as a result, effectively inhibits aggregation of thrombocytes [Askew, B. C., et al., J. Med. Chem. 1997, 40(12):1779], while [6S-[6α[R*(trans),8aα]]]-N-[1-(4-aminocyclohexyl)-2-(2-benzothiazolyl)-2-oxoethyl]-1,4-dioxo-2-(3-phenylpropyl)perhydro-pyrrolo[1,2-α]pyrazine-6-carboxamide is an anticoagulant, which inhibits serine thrombin and factor Xa proteases [Berryman, K. A.; Doherty, A. M.; Edmunds, J. J.; Siddiqui, M. A. Pfizer Inc. WO 9748706, 1997].

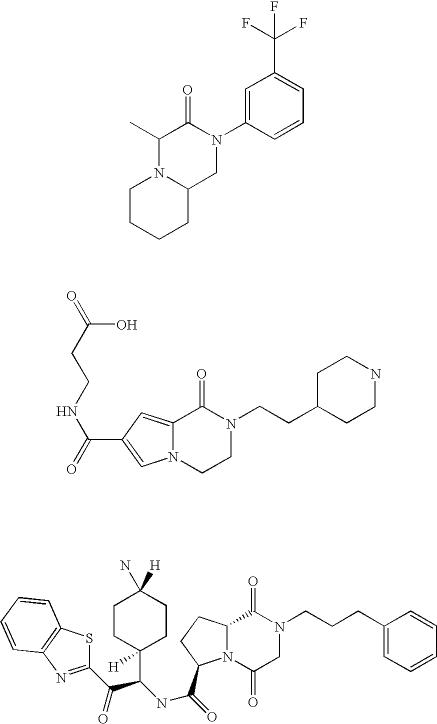

Antibacterial properties are exhibited by 3-isopropyl-hexahydropyrrolo[1,2-a]pyrazine-1,4-dione [Kwon O., et. al. J. Antibiot. 2001, 54(2): 179], and N-[4-(3-oxo-3,5-dihydro-1,4,8b-triaza-acenaphthylen-4-yl)butyl]trifluoromethane-sulfonamide is an inhibitor of receptor PDGF kinase and, for this reason, a potential drug to treat renal insufficiency, hyperlipidemia, and hypertension [Ikemoto, T., et al. Tetrahedron 2000, 56(40): 7915]. High antiprotozoic activity is exhibited by 3,4,7,8-tetrahydro-2H-pyrazino[1,2-a]indole-1,6,9-trione [Tapia, R., et al. Eur. J. Org. Chem. 2002, 23: 4005], and 2-{4-[4-(3-trifluoromethylphenyl)piperazin-1-yl]butyl}hexahydropyrido[1,2-α]pyrazine-1,4-dione is a highly effective ligand of serotonin 5-HTla receptor [Lopez-Rodriguez, M., et al. J. Med. Chem. 2001, 44(2): 186].

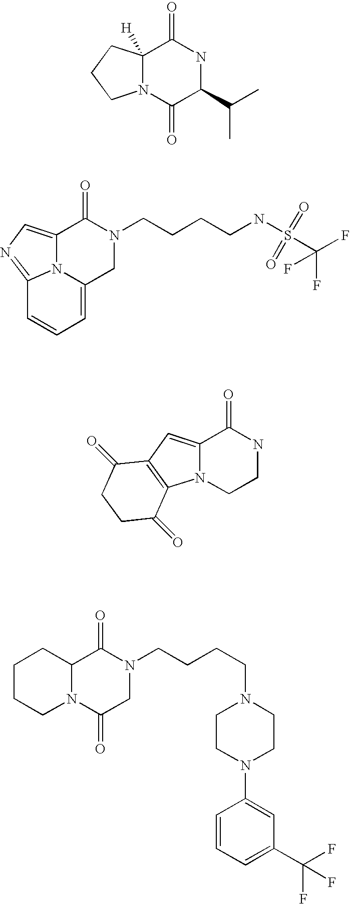

Considering the high potential and broad spectrum of physiological activity of annellated aza-heterocyclic compounds, there is increasing urgency to develop new compounds of this type, focused libraries, and pharmaceutical compositions including these compounds, and also methods of preparing and using the same.

As a result of studies conducted to find new physiologically active compounds and leader compounds, these inventors have produced previously unknown annellated carbamyl-aza-heterocyclic compounds exhibiting physiological activity, a focused library, and a pharmaceutical composition containing these compounds, and developed methods of preparing and using the same.

### DISCLOSURE OF THE INVENTION

The terms used in the description of the invention are defined below.

“Combinatorial library” is a collection of compounds produced by parallel synthesis and intended for searching a hit or leader compound, and also for optimizing the physiological activity of the hit or leader, each compound in the library corresponding to the common scaffold, the library being a collection of related homologues or analogues.

“Focused library” is a combinatorial library or a combination of several combinatorial libraries, or a combination of libraries and compounds organized in a special way to enhance the probability of finding hits and leaders or to improve the efficiency of their optimization. The design of focused libraries is, as a rule, linked to the direction in which a search is conducted for effectors (inhibitors, activators, agonists, antagonists, and so on) of specific bio-targets (enzymes, receptors, ion channels, and so on).

“Hit compound” or “hit” is a compound that exhibited desired physiological activity during primary screening.

“TLeader comnpound” or “leader” is a compound of an outstanding (maximum) physiological activity associated with a specific bio-target related to a particular (or several) pathology or disease.

“Parallel synthesis” is a method of conducting combinatorial chemical synthesis of the library.

“Scaffold” is a general structural formula or molecular framework, or an invariant region of compounds typical of all compounds comprising the combinatorial library.

“Nucleophilic” means electron-excessive reagent.

“Electrophilic” means an electron-deficient reagent.

“Substituent” means a chemical radical that is attached to the scaffold or synthesis intermediate during the synthesis thereof, for example, “nucleophilic substituent,” “electrophilic substituent,” “NH-protective substituent,” or “inert substituent.”

“Optionally substituted radical” means a radical without substituents or a radical containing one or several substituents, including “nucleophilic substituent,” “electrophilic substituent,” “NH-protective substituent,” and/or “inert substituent.”

“Nucleophilic substituent” is a chemical radical that is attached to the scaffold as a result of reaction with a nucleophilic reagent, for example, one selected from a group of primary or secondary amines, alcohols, phenols, mercaptans, and thiophenols.

“Electrophilic substituent” means a chemical radical that is attached to the scaffold as a result of reaction with an electrophilic reagent, for example, one selected from a group of organic halides (optionally substituted C₁-C₇ alkyl halides, optionally substituted aryl C₁-C₇ alkyl halides, optionally substituted heterocyclyl-C₁-C₇ alkyl halides, optionally substituted aryl halides, optionally substituted heterocyclyl halides), organic acids or their derivatives (anhydrides, imidazolides, halo-anhydrides), esters of organic sulfo acids or organic sulfochlorides, organic haloformates, organic isocyanates, and organic isothio-cyanate aminomethyls.

“NH-protective substituent” means a chemical radical that is attached to the scaffold or synthesis intermediate for temporary protection of the amino group in multifunctional compounds, including, but not limited to, an amide substituent, such as formyl, optionally substituted acetyl (for example, trichloroacetyl, trifluoroacetyl 3-phenylpropionyl, etc.), optionally substituted benzoyl, etc.; carbamate substituent, such as optionally substituted C₁-C₇ alkyloxycarbonyl, for example, methyloxycarbonyl, ethyloxycarbonyl, tertbutyloxycarbonyl, 9-fluorenyl-methyloxycarbonyl (Fmoc), etc.; optionally substituted C₁-C₇ alkyl substituent, for example, tert-butyl, benzyl, 2,4-dimethyloxybenzyl, 9-phenylfluorenyl, etc.; sulfonyl substituent, for example, benzene sulfonyl, n-toluene sulfonyl, etc. NH-protective substituents are described in more detail in “Protective Groups in Organic Synthesis,” Third Fdition Greene, T. W. and Wuts, P. G. M. 1999, pp. 494-653. Published by John Wiley & Sons, Inc., New York, Chichester, Weinheim, Brisbane, Toronto, Singapore.

“Inert substituent” (or “non-interfering substituent”) means a low-reactive or non-reactive radical, including, but not limited to, C₁-C₇ alkyl, C₂-C₇ alkenyl, C₂-C₇ alkynyl, Ci-C₇ alkoxy, C₇-C₁₂ aralkyl, aralkyl substituted with inert substituents, C₇-C₁₂ heterocyclyl alkyl, heterocyclyl alkyl substituted with inert substituents, C₇-C₁₂ alkaryl, C₃-C₁₀ cycloalkyl, C₃-C₁₀ cycloalkenyl, phenyl, substituted phenyl, toluene, xylenyl, biphenyl, C₂-C₁₂ alkoxyalkyl, C₂-C₁₀ alkyl sulfinyl, C₂-C₁₀ alkyl sulfonyl, (CH₂)ₘ—O—(C₁-C₇ alkyl), —(CH₂)ₘ—N(C₁-C₇ alkyl)n, aryl, aryl substituted with inert substituents, alkoxy substituted with inert substituents, fluoroalkyl, aryloxyalkyl, heterocyclyl, heterocyclyl substituted with inert substituents, and nitroalkyl; wherein m and n vary from 1 to 7. Preferred “inert substituents” are C₁-C₇ alkyl, C₂-C₇ alkenyl, C₂-C₇ alkynyl, C₁-C₇ alkoxy, C₇-C₁₂ aralkyl, C₇-C₁₂ alkaryl, C₃-C₁₀ cycloalkyl, C₃-C₁₀ cycloalkenyl, C₁-C₇ alkyl substituted with inert substituents, phenyl, phenyl substituted with inert substituents, (CH₂)ₘ—O— (C₁-C₇ alkyl), —(CH₂)ₘ—N(C₁-C₇ alkyl)ₘ, aryl, aryl substituted with inert substituents, heterocyclyl, and heterocyclyl substituted with inert substituents.

“Aryl” means one or more aromatic cyclic compounds, each including 5 or 6 carbon atoms. “Aryl” may be a condensed polycyclic compound, for example, naphthalene, or uncondensed, for example, biphenyl. “Substituted aryl” has one or more substituents.

“Halogen” means an atom of fluorine, chlorine, bromine or iodine.

“Heterocyclie compound” means one or several saturated, unsaturated, or aromatic cyclic compounds having 5, 6 or 7 atoms, at least one of which is a heteroatom. Preferred heteroatoms are sulfur, oxygen, and nitrogen. A “heterocyclic compound” may be a condensed polycyclic compound, for example, benzimidazole, benzoxazole, benzothiazole, and quinoline, or uncondensed, for example, bipyridyl.

“Azoheterocyclic compound” means a heterocyclic compound containing at least one nitrogen atom, for example, piperidine, morpholine, pyrrole, benzimidazole, benzoxazole, benzothiazole, and quinoline.

“Substituted heterocyclic compound” means a heterocyclic compound having one or several substituents.

“Substituted azoheterocyclic compound” means an azoheterocyclic compounds having one or several substituents.

An object of this invention is to produce novel annellated carbamyl-aza-heterocyclic compounds.

This object is achieved by using annellated carbamyl-aza-heterocyclic compounds of general formula 1: 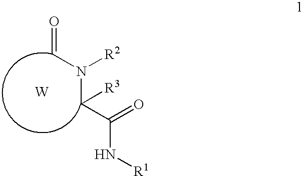
wherein:
W is a 6-oxopiperazine, [1,4]diazepan, [1,4]thiazepan or [1,4]oxazepan cyclic ring annellated with at least one optionally substituted and one optionally condensed heterocyclic compound Q;
Q is a pyrrole, pyrazole, imidazole, thiazole, pyrrolidine, indole, benzofuran, 4,5,6,7-tetrahydrobenzothiophene, thieno [3,2-b]pyrrole, furo[3,2-b]pyrrole, thieno[2,3-b]pyrrole, benzimidazole, pyridine, quinoline or 1,2,3,4-tetrahydro-isoquinoline cyclic compound; and
R¹, R² and R³ are, independently of one another, a hydrogen atom, an inert substituent, an optionally substituted C₁-C₆ alkyl, an optionally substituted C₃-C₈ cycloalkyl, an optionally substituted phenyl, an optionally substituted aryl, or an optionally substituted heterocyclyl.

According to the invention, the preferred annellated carbamyl-aza-heterocyclic compounds are substituted 1-oxo-1,2,3,4-tetrahydropyrrolo[1,2-a]pyrazine-3-carboxamides of general forrnula 1.1, 3-oxo-2,3,4,6-tetrahydro-1H-pyrrolo[1,2-a]pyrazine-1-carboxamides of general formula 1.2, 6-oxo-5,6-dihydro-4H-benzo[f]pyrrolo[1,2-a] [1,4]diazepine-4-carboxamides of general formula 1.3, and 5-oxo-1,3,4,5,6,7,8,9-octahydro-2H-10-thia-6,9-diaza-benzo[a]-cyclopenta[e]azulene-7-carboxamides of general formula 1.4: 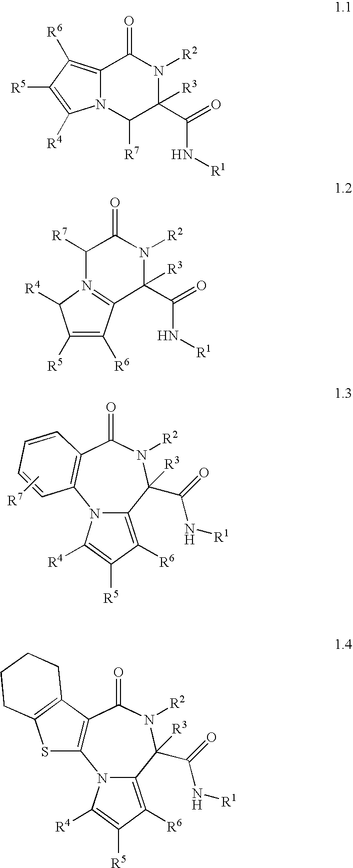
wherein:
R¹, R² and R³ are as above;
R⁴, R⁵ and R⁶ are, independently of one another, a hydrogen atom, an inert substituent, an optionally substituted C₁-C₆ alkyl, an optionally substituted C₃-C₈ cycloalkyl, an optionally substituted phenyl, an optionally substituted aryl, or an optionally substituted heterocyclyl; and
R⁷ is a hydrogen atom or an optionally substituted C₁-C₆ alkyl.

According to the invention, the preferred annellated carbamyl-aza-heterocyclic compounds also are substituted 4-oxo-4,5,6,7-tetrahydropyrazolo[1,5-a]pyrazine-6-carboxamides of general formula 1.5, 4-oxo-1,4,5,6-tetrahydro-1,2,5,9a-tetra-aza-cyclopenta[e]azulene-6-carboxamides of general formula 1.6, and 6-oxo-4,5,6,7-tetrahydro-1H-8-thia-1,2,5-triaza-azulene-4-carboxamides of general formula 1.7: 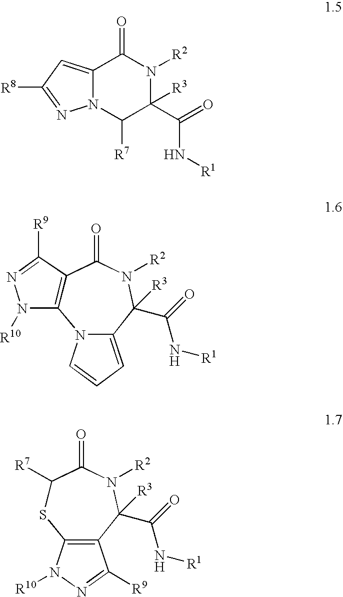
wherein:
R¹, R², R³, and R⁷ are as above;
R⁸ and R⁹ are, independently of one another, an inert hydrogen atom, carboxyalkyl, carboxy, or an optionally substituted carbamyl; and
R⁸, R⁹ and R¹⁰ are, independently from one another, an inert substituent, an optionally substituted C₁-C₆ alkyl, an optionally substituted C₃-C₈ cycloalkyl, an optionally substituted phenyl, an optionally substituted aryl, or an optionally substituted heterocyclyl.

According to the invention, the preferred annellated carbamyl-aza-heterocyclic compounds also are substituted 8-oxo-5,6,7,8-tetrahydro-imidazo[1,5-a]pyrazine-6-carboxamides of general formula 1.8: 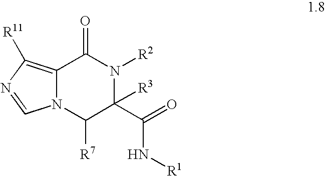
wherein:
R¹, R², R³ and R⁷ are as above; and
R¹¹ is a hydrogen atom, carboxyalkyl, carboxy, or an optionally substituted carbamyl.

According to the invention, the preferred annellated carbamyl-aza-heterocyclic compounds also are substituted 8-oxo-5,6,7,8-tetrahydro-4-oxa-1-thia-3,7-diaza-azulene-6-carboxamides of general formula 1.9: 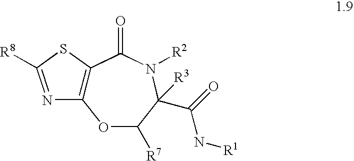
wherein: R¹, R², R³, R⁷ and R⁸ are as above.

According to the invention, the preferred annellated carbamyl-aza-heterocyclic compounds also are substituted 1-oxo-1,2,3,4-tetrahydropyrazino[1,2-a]indole-3-carboxamides of general formula 1.10, 7-oxo-5,7,8,10-tetrahydro-6H-9-thia-6, 10-diaza-benzo[a]azulene-5-carboxamides of general formula 1.11, 7-oxa-7,8,9,10-tetrahydro-6H-5-thia-8, 10-diaza-benzo[a]azulene-9-carboxamides of general formula 1.12, and 9-oxo-7,8,9,10- tetrahydro-6H-5-thia-8,10-diaza-benzo[a]azulene-7-carboxamides of general formula 1.13: 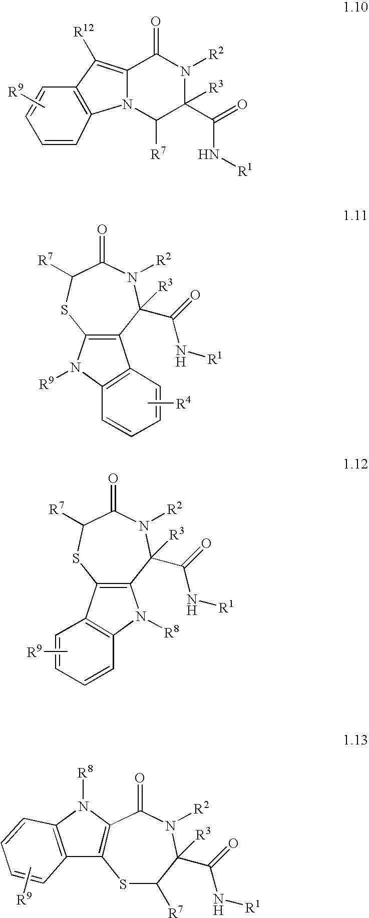
wherein:
R¹, R², R³, R⁷, R⁸ and R⁹ are as above; and
R¹² is a hydrogen atom, an inert substituent, a substituted amino group, or pyrrol-1-yl.

According to the invention, the preferred annellated carbamyl-aza-heterocyc lic compounds also are substituted 9-oxo-6,7,8,9-tetrahydro-10-ox a-5-thia-8-aza-benzo[a] azulene-7-carboxamides of general formula 1.14: 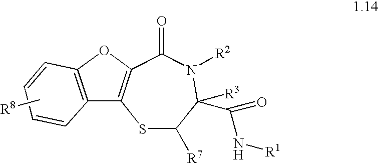
wherein: R¹, R², R³, R⁷ and R⁸ are as above.

According to the invention, the preferred annellated carbamyl-aza-heterocyclic compounds also are substituted 1-oxo-1,2,3,4-tetrahydro-benzo[4,5] imidazo[1,2-a]pyrazine-3-carboxamides of general formula 1.15: 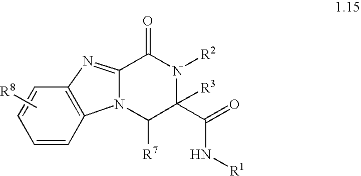
wherein: R¹, R², R³, R⁷ and R⁸ are as above.

According to the invention, the preferred annellated carbamyl-aza-heterocyclic compounds also are substituted 7-oxo-4,5,6,7-tetrahydro-1-thia-3b,6-diaza-cyclopenta[a]indene-5-carboxamides of general formula 1.16 and 7-oxo-4,5,6,7-tetrahydro-1-oxa-3b,6-diaza-cyclopenta[a]indene-5-carboxamides of general formula 1.17: 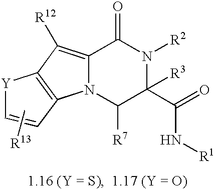
wherein:
R¹, R², R³, R⁷ and R¹² are as above; and
R¹³ is a hydrogen atom or an optionally substituted C₁-C₆ alkyl.

According to the invention, the preferred annellated carbamyl-aza-heterocyclic compounds also are substituted 7-oxo-4,5,6,7-tetrahydro-3-thia-3b,6-diaza-cyclopenta[a]indene-5-carboxamides of general formula 1.18: 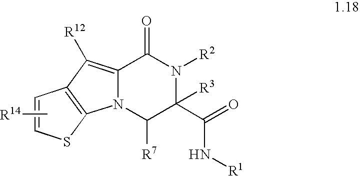
wherein:
R¹, R², R³, R⁷ and R¹² are as above; and
R¹⁴ is a hydrogen atom or an optionally substituted C₁-C₆ alkyl.

According to the invention, the preferred annellated carbamyl-aza-heterocyclic compounds also are substituted 7-oxo-5,6,7,8-tetrahydro-9-thia-1,6-diaza-benzocycloheptane-5-carboxamides of general formula 1.19 and 8-oxo-7,8,9,10-tetrahydro-6-thia-5,9-diaza-cyclopenta[b]naphthalene-10-carboxamides of general formula 1.20: 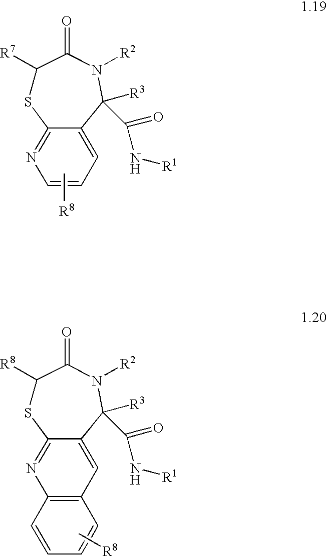
wherein: R¹, R², R³, R⁷ and R⁸ are as above.

According to the invention, the preferred annellated carbamyl-aza-heterocyclic compounds also are substituted 4-oxo-2,3,3a,4,5,6-hexahydro-1H-benzo[f]pyrrolo[1,2-a][1,4]diazepine-6-carboxamides of general formula 1.21, (3aS)-4-oxo-2,3,3a,4,5,6-hexahydro-1H-benzo[f]pyrrolo[1,2-a][1,4]diazepine-6-carboxamides of general formula 1.22, (3aS,6S)-4-oxo-2,3,3a,4,5,6-hexahydro-1H-benzo[f]pyrrolo[1,2-a][1,4]diazepine-6-carboxamides of general formula 1.22a, and (3aS,6R)-4-oxo-2,3,3a,4,5,6-hexahydro-1H-benzo[f]pyrrolo[1,2-a][1,4]diazepine-6-carboxamides of general formula 1.22b: 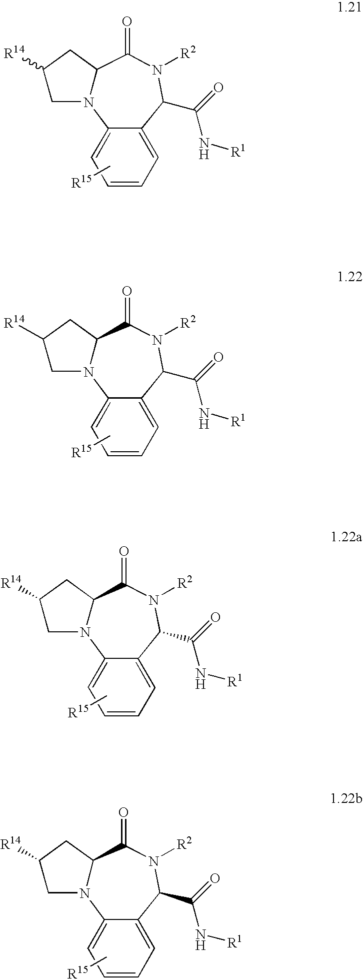
wherein:
R¹ and R² are as above;
R¹⁴ is a hydrogen atom or an optionally substituted hydroxyl group; and R¹⁵ is a hydrogen atom, NO₂, CF₃, CN, carboxyalkyl, carboxy, an optionally substituted carbamyl, or an optionally substituted amino group.

According to the invention, the preferred annellated carbamyl-aza-heterocyclic compounds also are substituted 1-oxo-1,3,4,6,11,11a-hexahydro-2H-pyrazino[1,2-b]isoquinoline-3-carboxamides of general formula 1.23 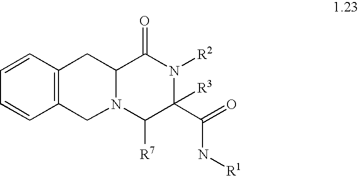
wherein: R¹, R², R³ and R⁷ are as above.

It is an object of this invention to develop a method for producing novel annellated carbamyl-aza-heterocyclic compounds of general formula 1.

This object is achieved by a method to produce novel annellated carbamyl-aza-heterocyclic compounds of general formula 1 by three-component condensation of a suitable isonitrile of general formula 2, a suitable primary amine of general forrnula 3, and a suitable heterocyclic bifunctional 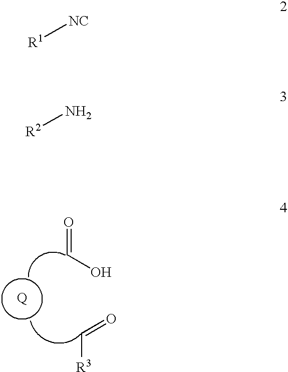
reagent of general fonnula 4:
wherein: Q, R¹, R² and R³ are as above.

According to the invention- preferred hifunctional reagents 4 are heterocyclic reagents containing a carboxyl and carbonyl groups of general formulas 4.1-4.23 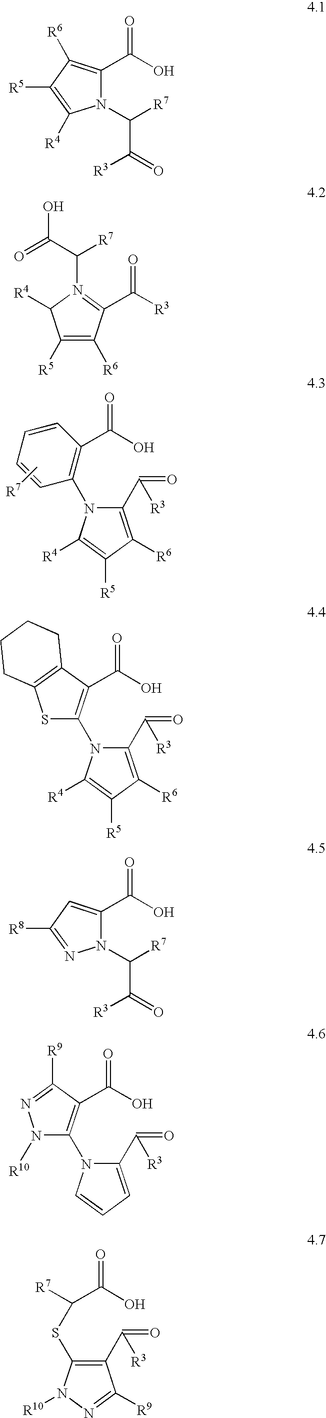 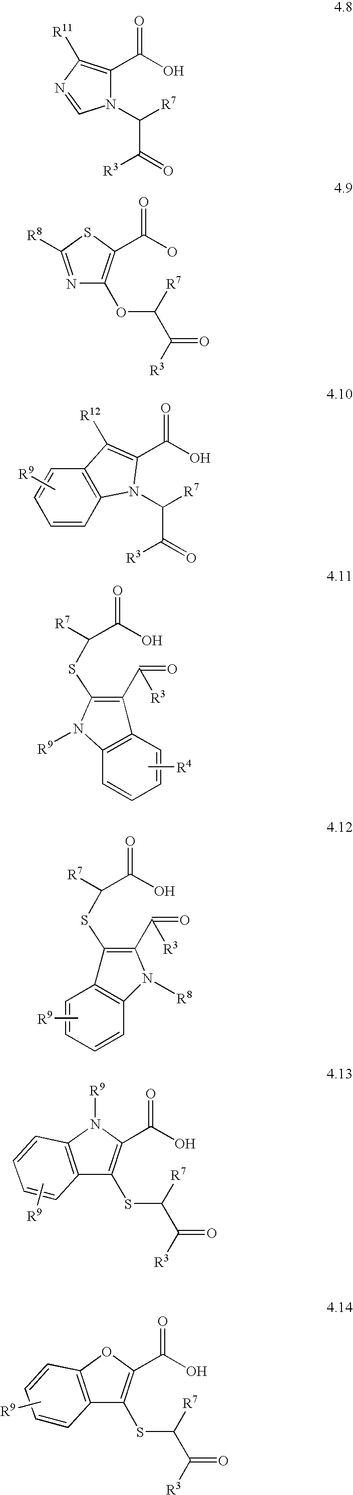 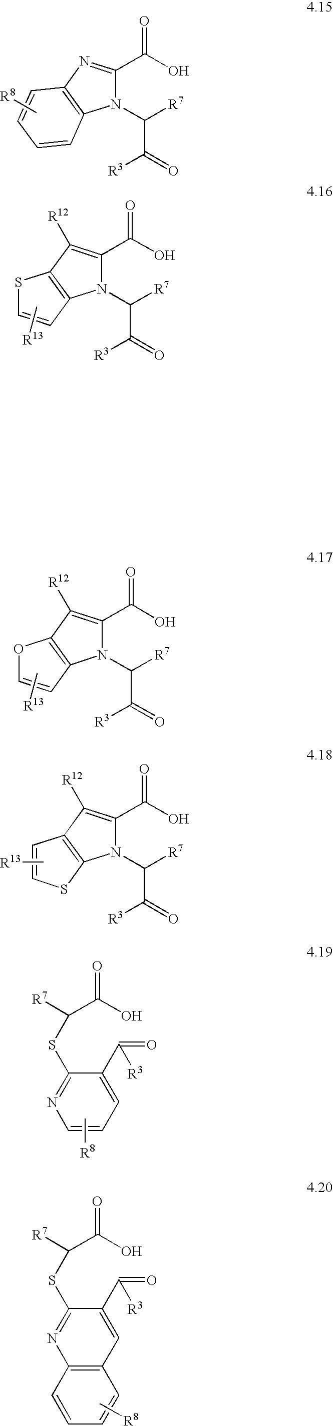 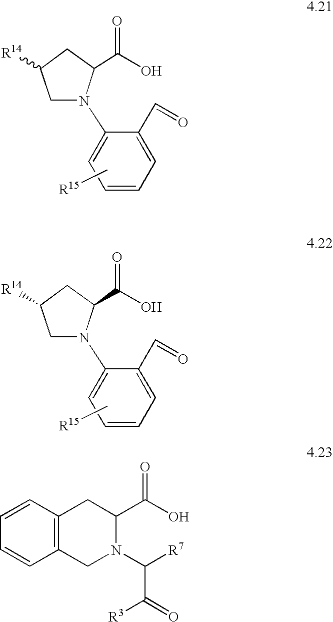
wherein: R³to R¹⁵ are as above.

Another object of this invention is to develop a novel focused library for determining leader compounds.

This object is achieved by developing a focused library designed to determine leader compounds and comprising at least one annellated carbamyl-aza-heterocyclic compound of general formula 1 as claimed in claim 1.

A further object of this invention is to produce a novel pharmaceutical composition exhibiting anticancer activity.

This object is achieved by producing a pharmaceutical composition exhibiting anticancer activity, which comprises at least one annellated carbamyl-aza-heterocyclic compound of general formula 1 or a pharmaceutically acceptable salt thereof.

### BEST EMBODIMENT OF THE INVENTION

The invention is described below with reference to examples, which illustrate, but do not limit, the scope of this invention.

### EXAMPLE 1

A general method of producing annellated carbamyl-aza-heterocyclic compounds of general formula 1.

A mixture of 3 mmol of a suitable isonitrile of general formula 2, 3 mmol of a suitable primary amine of general formula 3, and 3.1 mmol of a suitable bifunctional reagent of general formula 4 was stirred in 5 ml of dry methanol or another suitable solvent at room temperature for 48 to 56 hours. The resulting residue was filtered off and, if necessary, was recrystallized from a suitable solvent. This yielded compound 1 in the form of a mixture of enantiomers at a yield of 40% to 95% (Tables 1.01 to 1.23). In need, compound 1 was separated chromatographically into optical isomers (Tables 1.21-22).
Table 1.01(1). 1-oxo-6-(2-furyl)-1,2,3,4-tetrahydropyrrolo[1,2-a]pyrazine-3-carboxamides of general formula 1.1.
Substituted 1-oxo-6-(2-furyl)-1,2,3,4-tetrahydropyrrolo[1,2-a]pyrazine-3-carboxamides of general formula 1.1.
Table 1.01(2). Substituted 1-oxo-6-(2-phenyl)-1,2,3 ,4-tetrahydropyrrolo[1,2-a]pyrazine-3-carboxamides of general formula 1.1.
Table 1.02. Substituted 3-oxo-1,2,3,4-tetrahydropyrazino[1,2-a]indole-1-carboxamides of general formula 1.1.
Table 1.03. Substituted 6-oxo-5,6-dihydro-4H-benzo[f]pyrrolo [1,2-a] [1,4]diazepine-4-carboxamides of general formula 1.3.
Table 1.04. Substituted 5-oxo-1,3,4,5,6,7,8,9-octahydro-2H-10-thia-6,9-diaza-benzo[a]-cyclopenta[e]azulene-7-carboxamides of general formula 1.4.
Table 1.05(1). Substituted 5-aryl-2-tert-butyl-4-oxo-4,5,6,7-tetrahydropyrazolo[1,5-a]pyrazine-6-carboxamides of general formula 1.5.
Table 1.05(2). Substituted 5-benzyl-2-tert-butyl-4-oxo-4,5,6,7-tetrahydropyrazolo[1,5-a]pyrazine-6-carboxamides of general formula 1.5.
Table 1.05(3). Substituted 2-aryl-4-oxo-4,5 ,6,7-tetrahydropyrazolo[l ,5-a]pyrazi ne-6-carboxamides of general formula 1.5.
Table 1.05(4). Substituted 4-oxo-2-(2-furyl)-4,5,6,7-tetrahydropyrazolo[1,5-a]pyrazine-6-carboxamides of general formula 1.5.
Table 1.05(5). Substituted 2-carboxyalkyl-4-oxo-4,5,6,7-tetrahydropyrazolo[1,5-a]pyrazine-6-carboxamides of general formula 1.5.
Table 1.05(6). Substituted 2-carbamino-4-oxo-4,5,6,7-tetrahydropyrazolo[1,5-a]pyrazine-6-carboxamides of general formula 1.5.
Table 1.06. Substituted 4-oxo-1,4,5 ,6-tetrahydro-1,2,5,9a-tetra-aza-cyclopenta[e]azulene-6-carboxamides of general formula 1.6.
Table 1.07. Substituted 6-oxo-4,5 ,6,7-tetrahydro-1H-8-thia-1,2,5-triaza-azulene-4-carboxamides of general formula 1.7.
Table 1.08. Substituted ⁸-oxo-5,6,7,8-tetrahydroimidazo[1,5-a]pyrazine-6-carboxamides of general formula 1.8.
Table 1.09. Substituted 8-oxo-5,6,7,8-tetrahydro-4-oxa-1-thia-3,7-diaza-azulene-6-carboxamides of general formula 1.9.
Table 1.10(1). Substituted 7-methoxy- and 6,9-dimethoxy-1-oxo-1,2,3,4-tetrahydropyrazino[1,2-a]indole-3-carboxamides of general formula 1.10.
Table 1.10(2). Substituted 10-acetylamino-1-oxo-1,2,3,4-tetrahydropyrazino[1,2-a]indole-3-carboxamides of general formula 1.10.
Table 1.10(3). Substituted 1-oxo-10-pyrrol-1-yl)-1,2,3 ,4-tetrahydropyrazino[1,2-a]indole-3-carboxamides of general formula 1.10.
Table 1.11. Substituted 7-oxo-5 ,7,8,10-tetrahydro-6H-9-thia-6, 10-diaza-benzo[a]azulene-5-carboxamides of general formula 1.11.
Table 1.12. Substituted 7-oxo-7,8,9, 10-tetrahydro-6H-5-thia-8, 10-diaza-benzo[a]azulene-9-carboxamides of general formula 1.12.
Table 1.13. Substituted 9-oxo-7,8,9,10-tetrahydro-6H-5-thia-8,10-diaza-benzo[a]azulene-7-carboxamides of general formula 1.13.
Table 1.14. Substituted 9-oxo-6,7,8,9-tetrahydro-10-oxa-5-thia-8-aza-benzo[a]azulene-7-carboxamides of general formula 1.14.
Table 1.15. Substituted 1-oxo-1,2,3,4-tetrahydro-benzo[4,5]imidazo[1,2-a]pyrazine-3-carboxamides of general formula 1.15.
Table 1.16(1). Substituted 6-alkyl- and 6-cycloalkyl-7-oxo-4,5,6,7-tetrahydo-1-thia-3b,6-diaza-cyclopenta[a]indene-5-carboxamides of general formula 1.16.
Table 1.16(2). Substituted 6-aryl-7-oxo-4,5,6,7-tetrahydro-1-thia-3b,6-diaza-cyclopenta[a]indene-5-carboxamides of general formula 1.16.
Table 1.16(3). Substituted 6-benzyl-7-oxo-4,5,6,7-tetrahydro-1-thia-3b,6-diaza-cyclopenta[a]indene-5-carboxamides of general formula 1.16.
Table 1.16(4). Substituted 6-(2-hydroxyethyl)- and 6-(2-aminoethyl)-7-oxo-4,5,6,7-tetrahydro-1-thia-3b,6-diaza-cyclopenta[a]indene-5-carboxamides of general formula 1.16.
Table 1.16(5). Substituted 6-(3-hydroxypropyl)- and 6-(3-aminopropyl)-7-oxo-4,5,6,7-tetrahydro-1-thia-3b,6-diaza-cyclopenta[a]indene-5-carboxamides of general formula 1.16.
Table 1.16(6). Substituted 7-oxo-5-phenyl-4,5,6,7-tetrahydro-1-thia-3b,6-diaza-cyclopenta[a]indene-5-carboxamides of general formula 1.16.
Table 1.17(1). Substituted 6-alkyl- and 6-cycloalkyl-7-oxo-4,5,6,7-tetrahydro-1-oxa-3b,6-diaza-cyclopenta[a]indene-5-carboxamides of general formula 1.17
Table 1.17(2). Substituted 6-aryl-7-oxo-4,5,6,7-tetrahydro-1-oxa-3b,6-diaza-cyclopenta[a]indene-5-carboxamides of general formula 1.17
Table 1.17(3). Substituted 6-benzyl-7-oxo-4,5,6,7-tetrahydro-1-oxa-3b,6-diaza-cyclopenta[a]indene-5-carboxamides of general formula 1.17
Table 1.17(4). Substituted 6-(2-hydroxyethyl)- and 6-(2-aminoethyl)-7-oxo-4,5,6,7-tetrahydro-1-oxa-3b,6-diaza-cyclopenta[a]indene-5-carboxamides of general formula 1.17
Table 1.17(5). Substituted 6-(3-hydroxypropyl)- and 6-(3-aminopropyl)-7-oxo-4,5,6,7-tetrahydro-1-oxa-3b,6-diaza-cyclopenta[a]indene-5-carboxamides of general formula 1.17
Table 1.17(6). Substituted 7-oxo-5-phenyl-4,5,6,7-tetrahydro-1-oxa-3b,6-diaza-cyclopenta[a]indene-5-carboxamides of general formula 1.17
Table 1.18. Substituted 7-oxo-4,5,6,7-tetrahydro-3-thia-3b,6-diaza-cyclopenta[a]indene-5-carboxamides of general formula 1.18.
Table 1.19-20. Substituted 7-oxo-5,6,7,8-tetrahydro-9-thia-1,6-diaza-benzocycloheptane-5-carboxamides of general formula 1.19 and 8-oxo-7,8,9, 10-tetrahydro-6-thia-5,9-diaza-cyclopenta[b]naphthalene-10-carboxamides of general formula 1.20.
Table 1.21-22. Substituted 4-oxo-2,3,3a,4,5,6-hexahydro-1H-benzo[f]pyrrolo[1,2-a][1,4]diazepine-6-carboxamides of general formula 1.21 and 1.22.
Table 1.23. Substituted 1-oxo-1,3,4,6, 11,1 la-hexahydro-2H-pyrazino[1,2-b]isoquinoline-3-carboxamides of general formula 1.23.

### EXAMPLE 2

Tests of biological activity of compounds of general formula 1.

A focused library was compiled to include compounds of general formula 1, as shown in Tables 1.01 to 1.23, and tested for anticancer activity. Anticancer activity of the compounds was measured by their capacity to kill cancerous cells in a tissue culture. Three cell lines representing three types of cancerous tumors were chosen, in particular: DLD-1 (adenocarcinoma of the rectum), DU-145 (carcinoma of the brain), and T-47D (tumor of the mammary gland). All the cell cultures were received from the American Tissue Culture Collection (ATCC). The efficiency of the compounds was assessed by measuring the number of dead cells following treatment with the test compounds over 48 hours. The cells were seeded at a concentration of 5*10³ cells per hole of a standard 96-hole plate and left overnight for cells to be attached to the hole bottom. Whereupon the test compounds were added to the cells at an ultimate concentration of 30 μM, and the plates were left for 48 hours in an incubator at 37° C. maintaining the 5% CO₂/95% air atmosphere at 100% humidity. At the end of the experiment, the medium with the compounds was sucked out of all the holes, and the holes were rinsed twice with physiological solution. 50 μM of Alamar Blue solution was added to each hole, and fluorescence (λex=531 nm, λem=589 nm) was measured immediately after addition and 2 hours thereafter. The rate of fluorescence growth was proportional to the number of cells that survived incubation with the test compounds [John O'Brien, Ian Wilson, Terry Orton and Francois Pognan, “Investigation of the Alamar Blue (resazurin) Fluorescent Dye for the Assessment of Mammalian Cell Cytotoxicity.” Eur. J. Biochem. 2000,b 267, 5421-5426]. The proportion of cell growth suppression was found from the following equation:
% of growth inhibition=((ΔΦₖ−ΔΦᵢ)/ΔΦₖ)*100%,
wherein: ΔΦ means increase in fluorescence over two years following addition of the Alamar Blue solution, and the subscripts A and i mean, respectively, control cells (grown without any compounds added) and experimental cells (grown in the presence of test compounds). Data for some of the test compounds are given in Table 24. It follows from Table 24 that test compounds exhibit a high activity in suppressing tumor cell growth. What is more, compounds 1.5(3){44}, 1.5(3){50}, 1.10 (1){18}, and 1.16 (2){28} are equally good at suppressing cell growth in all three cancer tumors, while the remaining compounds show a varying degree of selectivity toward different tumors.

### EXAMPLE 3

A method of producing a pharmaceutical composition in tablets.

A mixture is prepared from 800 mg of starch, 800 mg of ground lactose, 200 mg of talc, and 500 mg of cyclohexylamide of 6-methyl-4-oxo-5-(2-pyrrolidin-1-yl-ethyl)-2-(4-ethylphenyl)-4,5,6,7-tetrahydropyrazolo[1,5-a]pyrazine-6-carbonic acid 1.5(3){50} and pressed into a bar. The bar thus produced is crushed into granules and screened so that 14 to 16 mesh granules could be collected. The granules are then compressed into tablets of suitable shape weighing 280 mg each. Similarly, pharmaceutical compositions are obtained in the form of tablets containing other 1,3-dioxo-2,3-dihydro-1H-pyrrolo[3,4-c]quinolines of general formula 1 as the active ingredient.

### EXAMPLE 4

A method of producing a pharmaceutical composition in capsules.

Compound 1.5(3){50} is carefully mixed with powdered lactose at a ratio of 2:1, and the resulting mixture is packed into gelatin capsule of suitable size 300 mg to a capsule.

### EXAMPLE 5

A method of producing a pharmaceutical composition for intramuscular, intraperitoneal, or subcutaneous injections.

A hydrochloride of compound 1.5(3){50} is mixed with 300 mg of chlorobutanol, 2 ml of propylene glycol, and 100 ml of injection water. The resultant solution is filtered and placed in 1 ml ampoules, which are sealed and sterilized in an autoclave.

### Commercial Applicability

This invention may be used in medicine, veterinary, biochemistry, and general chemistry.

LENGTHY TABLE REFERENCED HERE
US20070191337A1-20070816-T00001
Please refer to the end of the specification for access instructions.

LENGTHY TABLE REFERENCED HERE
US20070191337A1-20070816-T00002
Please refer to the end of the specification for access instructions.

LENGTHY TABLE REFERENCED HERE
US20070191337A1-20070816-T00003
Please refer to the end of the specification for access instructions.

LENGTHY TABLE REFERENCED HERE
US20070191337A1-20070816-T00004
Please refer to the end of the specification for access instructions.

LENGTHY TABLE REFERENCED HERE
US20070191337A1-20070816-T00005
Please refer to the end of the specification for access instructions.

LENGTHY TABLE REFERENCED HERE
US20070191337A1-20070816-T00006
Please refer to the end of the specification for access instructions.

LENGTHY TABLE REFERENCED HERE
US20070191337A1-20070816-T00007
Please refer to the end of the specification for access instructions.

LENGTHY TABLE REFERENCED HERE
US20070191337A1-20070816-T00008
Please refer to the end of the specification for access instructions.

LENGTHY TABLE REFERENCED HERE
US20070191337A1-20070816-T00009
Please refer to the end of the specification for access instructions.

LENGTHY TABLE REFERENCED HERE
US20070191337A1-20070816-T00010
Please refer to the end of the specification for access instructions.

LENGTHY TABLE REFERENCED HERE
US20070191337A1-20070816-T00011
Please refer to the end of the specification for access instructions.

LENGTHY TABLE REFERENCED HERE
US20070191337A1-20070816-T00012
Please refer to the end of the specification for access instructions.

LENGTHY TABLE REFERENCED HERE
US20070191337A1-20070816-T00013
Please refer to the end of the specification for access instructions.

LENGTHY TABLE REFERENCED HERE
US20070191337A1-20070816-T00014
Please refer to the end of the specification for access instructions.

LENGTHY TABLE REFERENCED HERE
US20070191337A1-20070816-T00015
Please refer to the end of the specification for access instructions.

LENGTHY TABLE REFERENCED HERE
US20070191337A1-20070816-T00016
Please refer to the end of the specification for access instructions.

LENGTHY TABLE REFERENCED HERE
US20070191337A1-20070816-T00017
Please refer to the end of the specification for access instructions.

LENGTHY TABLE REFERENCED HERE
US20070191337A1-20070816-T00018
Please refer to the end of the specification for access instructions.

LENGTHY TABLE REFERENCED HERE
US20070191337A1-20070816-T00019
Please refer to the end of the specification for access instructions.

LENGTHY TABLE REFERENCED HERE
US20070191337A1-20070816-T00020
Please refer to the end of the specification for access instructions.

LENGTHY TABLE REFERENCED HERE
US20070191337A1-20070816-T00021
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070191337A1). An electronic copy of the table will also be available from the USPTO upon request and payment of the fee set forth in 37 CFR 1.19(b)(3).

What is claimed is:

1. Annellated carbamyl-aza-heterocyclic compounds of general formula 1: 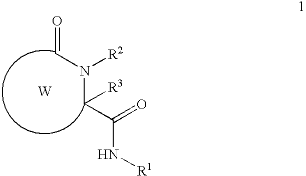
wherein:
W is a 6-oxopiperazine, [1,4]diazepan, [1,4]thiazepan, or [1,4]oxazepan cyclic compound annellated by at least one optionally substituted and optionally condensed heterocyclic compound Q;
R¹, R² and R³ are, independently of one another, a hydrogen atom, an inert substituent, an optionally substituted C₁-C₆ alkyl, an optionally substituted C₃-C₈ cycloalkyl, an optionally substituted phenyl, an optionally substituted aryl, or an optionally substituted heterocyclyl; and
Q is a pyrrole, pyrazole, imidazole, thiazole, pyrrolidine, indole, benzofuran, 4,5,6,7-tetrahydro-benzothiophene, thieno[3,2-b]pyrrole, furo[3,2-b]pyrrole, thieno[2,3-b]pyrrole, benzimidazole, pyridine, quinoline, or 1,2,3,4-tetrahydro-isoquinoline cyclic compound.

2. Compounds of claim 1, which are substituted 1-oxo-1,2,3,4-tetrahydropyrrolo[1,2-a]pyrazine-3-carboxamides of general formula 1.1 and 3-oxo-2,3,4,6-tetrahydro-1H-pyrrolo[1,2-a]pyrazine-1-carboxamides of general formula 1.2,6-oxo-5,6-dihydro-4H-benzo[q]pyrrolo[1,2-a][1,4]diazepine-4-carboxamides of general formula 1.3, 5-oxo-1,3,4,5,6,7,8,9-octahydro-2H-10-thia-6,9-diaza-benzo[a]-cyclopenta[e]azulene-7-carboxamides of general formula 1.4: 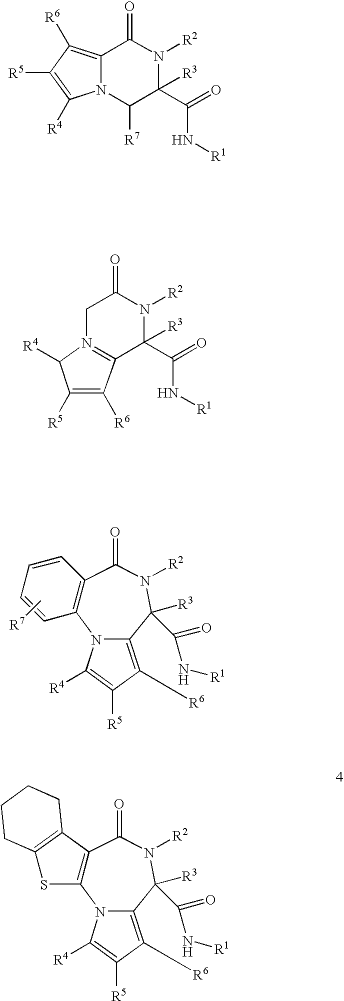
wherein:
R¹, R² and R³ are as above;
R⁴, R⁵ and R⁶ are, independently of one another, a hydrogen atom, an inert substituent, an optionally substituted C₁-C₆ alkyl, an optionally substituted C₃-C₈ cycloalkyl, an optionally substituted phenyl, an optionally substituted aryl, or an optionally substituted heterocyclyl; and
R⁷ is a hydrogen atom or an optionally substituted C₁-C₆ alkyl.

2. Compounds of claim 1, which are substituted 4-oxo-4,5,6,7-tetrahydropyrazolo[1,5-a]pyrazine-6-carboxamides of general formula 1.5, 4-oxo-1,4,5,6-tetrahydro-1,2,5,9a-tetra-aza-cyclopenta[e]azulene-6-carboxamides of general formula 1.6 and 6-oxo-4,5,6,7-tetrahydro-1H-8-thia-1,2,5-triaza-azulene-4-carboxamides of general formula 1.7: 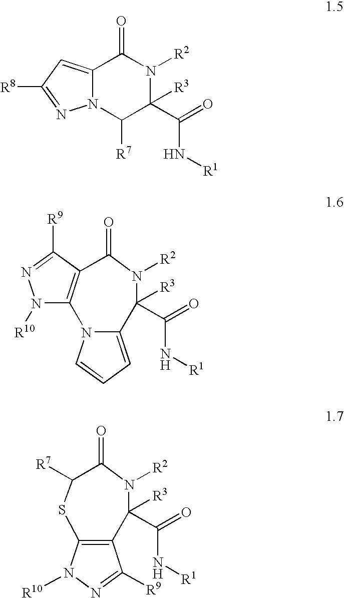
wherein:
R¹, R², R³, and R⁷ are as above;
R⁸ and R⁹ are, independently of one another, a hydrogen atom, carboxyalkyl, carboxy, or an optionally substituted carbamyl; and
R⁸, R⁹ and Rlo are, independently of one another, an inert substituent, an optionally substituted C₁-C₆ alkyl, an optionally substituted C₃-C₈ cycloalkyl, an optionally substituted phenyl, an optionally substituted aryl, or an optionally substituted heterocyclyl.

4. Compounds of claim 1, which are substituted 8-oxo-5,6,7,8-tetrahydro-imidazo[1,5-a]pyrazine-6-carboxamides of general formula 1.8: 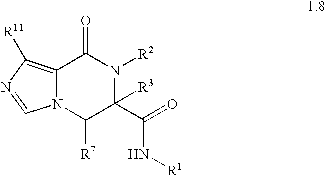
wherein:
R¹, R², R³ and R⁷ are as above; and
R¹¹ is a hydrogen atom, carboxyalkyl, carboxy, or an optionally substituted carbamyl.

5. Compounds of claim 1, which are substituted 8-oxo-5,6,7,8-tetrahydro-4-oxa-1-thia-3,7-diaza-azulene-6-carboxamides of general formula 1.9: 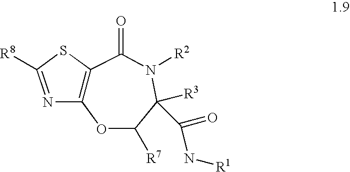
where: R¹, R², R³, R⁷ and R⁸ are as above.

6. Compounds of claim 1, which are substituted 1-oxo-1,2,3,4-tetrahydropyrazine[1,2-a]indole-3-carboxamides of general formula 1.10, 7-oxo-5,7,8,10-tetrahydro-6H-9-thia-6,10-diaza-benzo[a]azulene-5-carboxamides of general formula 1.11, 7-oxo-7,8,9,10-tetrahydro-6H-5-thia-8,10-diaza-benzo[a]azulene-9-carboxamides of general formula 1.12, and 9-oxo-7,8,9, 10-tetrahydro-6H-5-thia-8, 10-diaza-benzo[a]azulene-7-carboxamides of general formula 1.13: 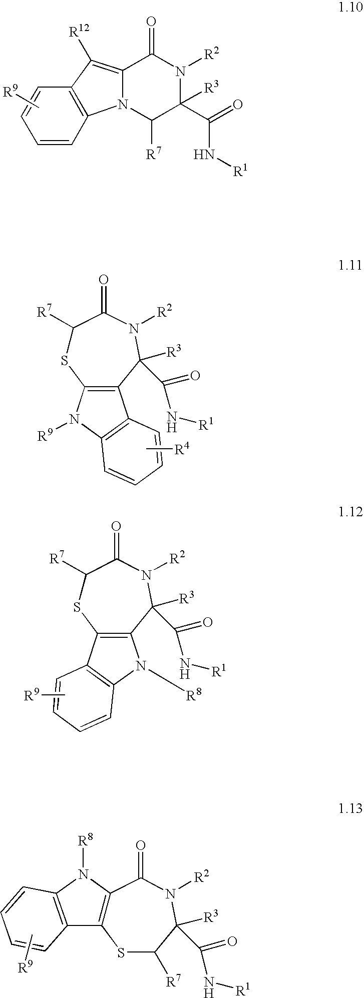
wherein:
R¹, R², R³, R⁷, R⁸, and R⁹ are as above; and
R¹² is a hydrogen atom, an inert substituent, a substituted amino group, or pyrrol-1-yl.

7. Compounds of claim 1, which are substituted 9-oxo-6,7,8,9-tetrahydro-10-oxa-5-thia-8-aza-benzo[a]azulene-7-carboxamides of general formula 1.14: 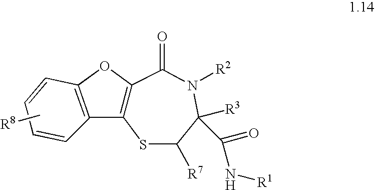
wherein: R¹, R², R³, R⁷ and R⁸ are as above.

8. Compounds of claim 1, which are substituted 1-oxo-1,2,3,4-tetrahydro-benzo[4,5]imidazo[1,2-a]pyrazine-3-carboxamides of general formula 1.15: 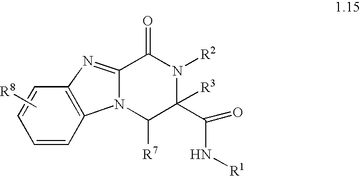
wherein: R¹, R², R³, R⁷ and R⁸ are as above.

9. Compounds of claim 1, which are substituted 7-oxo-4,5,6,7-tetrahydro-1-thia-3b,6-diaza-cyclopenta[a]indene-5-carboxamides of general formula 1.16 and 7-oxo-4,5,6,7-tetrahydro-1-oxa-3b,6-diaza-cyclopenta[a]indene-5-carboxamides of general formula 1.17: 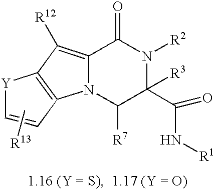
wherein:
R¹, R², R³, R⁷ and R¹² are as above; and
R¹³ is a hydrogen atom or an optionally substituted C₁-C₆ alkyl.

10. Compounds of claim 1, which are substituted 7-oxo-4,5,6,7-tetrahydro-3-thia-3b,6-diaza-cyclopenta[a]indene-5-carboxamides of general formula 1.18: 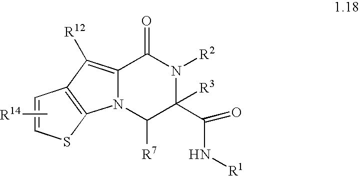
wherein:
R¹, R², R³, R⁷ and R¹² are as above; and R¹⁴ is a hydrogen atom or an optionally substituted C₁-C₆ alkyl.

11. Compounds of claim 1, which are substituted 7-oxo-5,6,7,8-tetrahydro-9-thia-1,6-diaza-benzocycloheptane-5-carboxamides of general formula 1.19 and 8-oxo-7,8,9,10-tetrahydro-6-thia-5,9-diaza-cyclopenta[b]naphthalene-10-carboxamides of general formula 1.20: 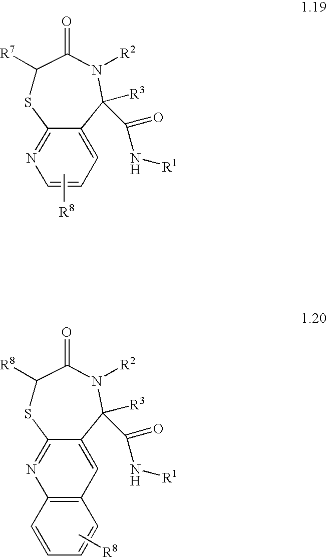
wherein: R¹, R², R³, R⁷ and R⁸ are as above.

12. Compounds of claim 1, which are substituted 4-oxo-2,3,3a,4,5,6-hexahydro-1H-benzo[f]pyrrolo[1,2-a][1,4]diazepin-6-carboxamides of general formula 1.21, (3aS)-4-oxo-2,3,3a,4,5,6-hexahydro-1H-benzo[f]pyrrolo[1,2-a] [1,4]diazepin-6-carboxamides of general formula 1.22, (3aS,6s)-4-oxo-2,3,3a,4,5,6-hexahydro-1H-benzo[f]pyrrolo[1,2-a] [1,4]diazepin-6-carboxamides of general formula 1.22a, and (3aS,6R)-4-oxo-2,3,3a,4,5,6-hexahydro-1H-benzo[f]pyrrolo[1,2-a][1,4]diazepin-6-carboxamides of general formula 1.22b: 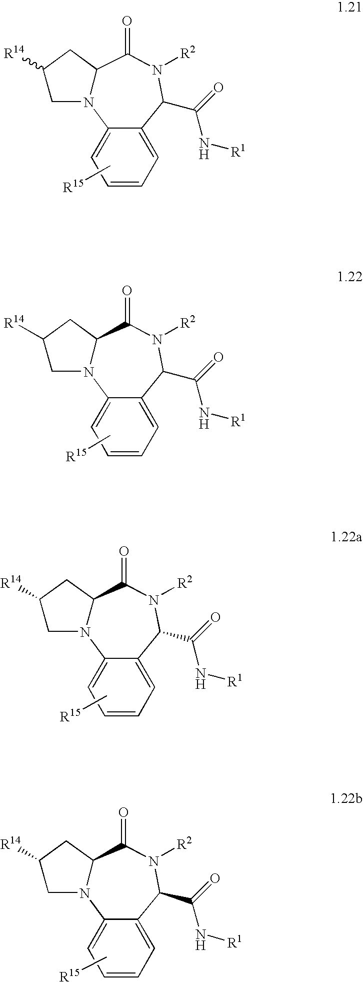
wherein:
R¹ and R² are as above;
R¹⁴ is a hydrogen atom or an optionally substituted hydroxyl group; and
R¹⁵ is a hydrogen atom, N0₂, CF₃, CN, carboxyalkyl, carboxy, an optionally substituted carbamyl, or an optionally substituted amino group.

13. Compounds of claim 1, which are substituted 1-oxo-1,3,4,6,11,1la-hexahydro-2H-pyrazino[1,2-b]isoquinoline-3-carboxamides of general formula 1.23: 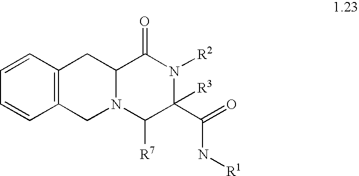
wherein: R¹, R², R³ and R⁷ are as above.

14. A method of producing annellated carbamyl-aza-heterocyclic compounds of general formula 1 by three-component condensation of a suitable isonitrile of general formula 2, a suitable primary amine of general formula 3, and a suitable heterocyclic bifunctional reagent of general formula 4: 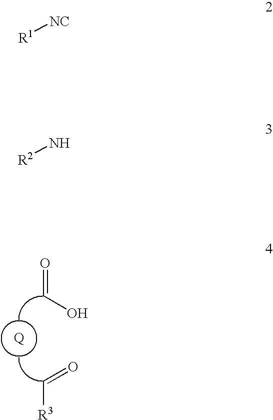
wherein: Q, R¹, R² and R³ are as above.

15. A method of producing compounds of claim 14, wherein bifunctional reagents 4 are heterocyclic compounds containing a carboxyl and a carbonyl groups of general formula 4.1-4.23: 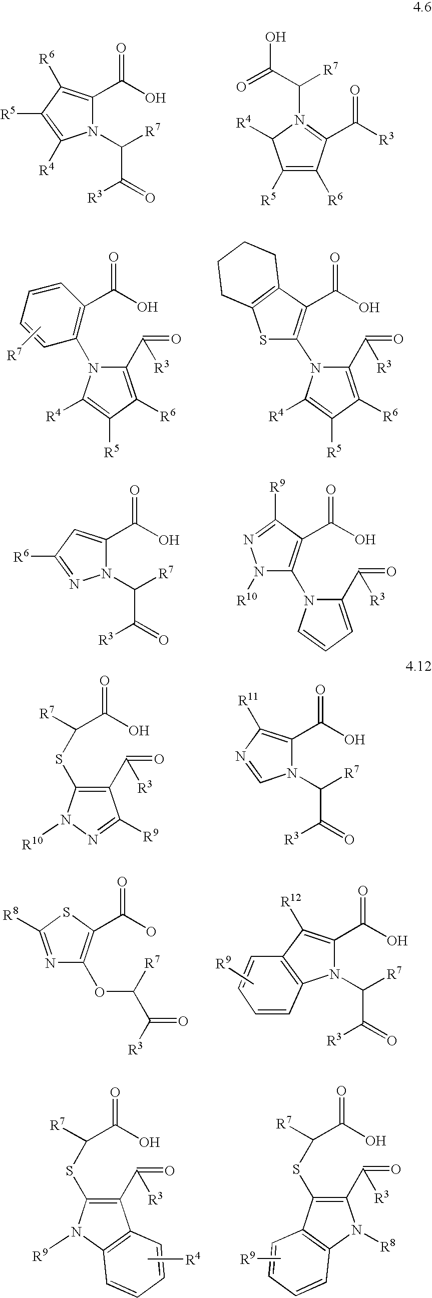 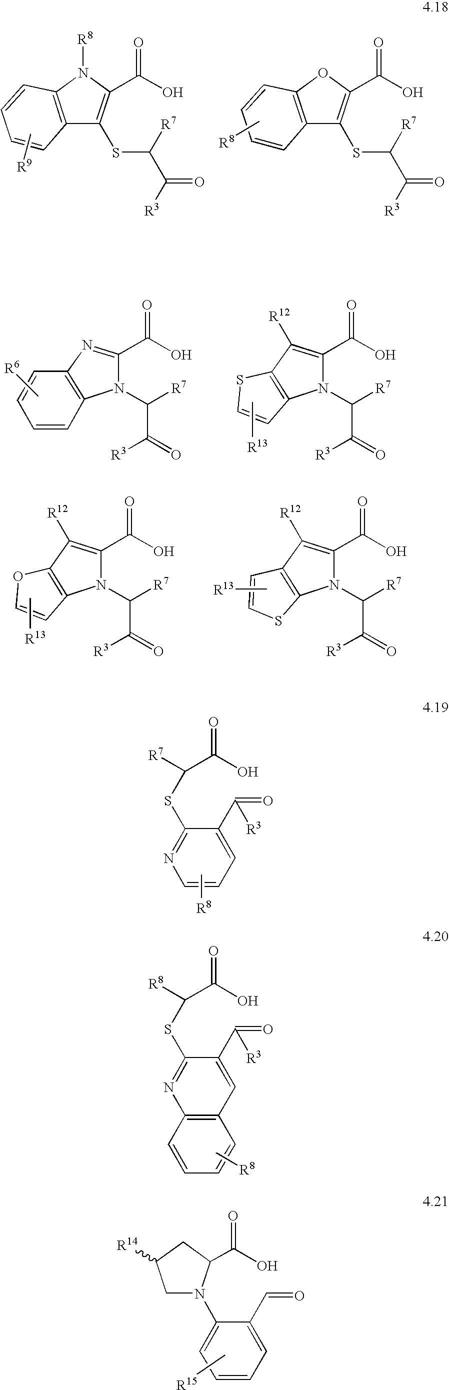 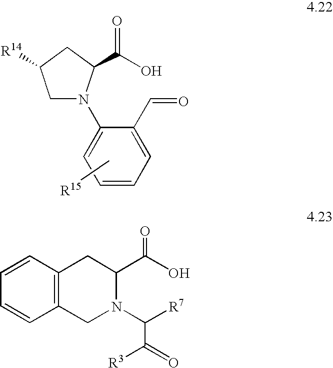
wherein: R³to R¹⁵ are as in claims 2 to 12.

15. A focused library for determining leader compounds, comprising at least one compound of general formula 1 according to claim 1.

16. A pharmaceutical composition exhibiting anticancer activity, comprising at least one annellated carbamyl-aza-heterocyclic compound of general formula 1 according to claim 1 or a pharmaceutically acceptable salt thereof.
